(19) United States
(12) Patent Application Publication
(10) Pub. No.: US 2012/0270205 A1
(43) Pub. Date: Oct. 25, 2012

(54) HIGH-FLUX CHEMICAL SENSORS

(76) Inventors: Sanjay V. PATEL, San Diego, CA (US); Marcel Benz, San Diego, CA (US)

(21) Appl. No.: 13/289,943

(22) Filed: Nov. 4, 2011

Related U.S. Application Data
(60) Provisional application No. 61/477,127, filed on Apr. 19, 2011.

(51) Int. Cl. C12Q 1/70; G01N 27/00; C12Q 1/02; G01N 30/96; C12M 1/34

(52) U.S. Cl. 435/5; 422/69; 435/287.7; 435/29; 436/149

(57) ABSTRACT

The present invention relates to the field of chemical detection. Specifically, the invention provides devices that respond quickly to various target chemical analytes present in the environment. Responses are based on a change in an electrical property (such as impedance or resistance) caused by adsorption or absorption of the target analyte(s) to or in a substrate-free chemical sensing element. The chemical sensing element is composed of a thin, electrically conductive polymer material (due to doping of structural polymer material(s) with electrically conductive particles and/or the use of electrically conductive polymer material(s)), which can allow vapors to pass through with little pressure drop. The chemical sensing material is either suspended in the environment, or emplaced adjacent to one or between two porous membranes, resulting in a sensing patch capable of high gas or vapor flux through the chemical sensing element.

### RELATED APPLICATION

This patent application claims the benefit of and priority to U.S. provisional patent application Ser. No. 61/477,127, filed 19 Apr. 2011 (attorney docket number SCS-4300-PV), which is hereby incorporated by reference in its entirety for any and all purposes.

### GRANT SUPPORT

The subject matter of this application was supported at least in part by U.S. Army Small Business Innovation Research (SBIR) grant no. W911QY-11-P-0051. The U.S. Government may have certain rights herein.

### BACKGROUND OF THE INVENTION

1. Field of the Invention

The present invention relates generally to the field of chemical detection and environmental monitoring. More specifically, the invention concerns devices that can detect one or more chemicals and/or biological materials in an environment as a result of their absorption or adsorption by one or more chemical sensing elements in the device, which alters a sensible electrical property of one or more electrode pairs in a circuit disposed in the device.

2. Background

The following description includes information that may be useful in understanding the present invention. It is not an admission that any such information is prior art, or relevant, to the presently claimed inventions, or that any publication specifically or implicitly referenced is prior art.

The ability to detect chemicals or biological materials in an environment is critically important in many contexts. For example, the detection of potential toxic chemicals in a home, place of business, industrial facility, or surrounding communities can prevent deaths, injuries, health problems in the event of accidents, fires, etc. The detection of unwanted chemicals or poisons in drinking water can alert users of the need to filter, purify, or treat the water before using to avoid adverse health consequences. It can also alert the water supplier of possible problems at the source or in the distribution system. Similarly, the detection of potentially harmful chemicals in lakes and other bodies of water can alert authorities to provide warnings to avoid consumption of fish and other fauna taken from the contaminated water source.

Further, the detection of chemicals and biological materials associated with explosives and chemical and biological warfare agents may be crucial in preventing acts of terrorism. Early detection of tell-tale chemicals or biological materials can provide the opportunity to warn the public and, if warranted, allow evacuation of at risk areas and populations.

The accurate detection of certain chemicals is also important in many industrial settings. For example, many products and components, such as computer chips and certain medical devices, must be manufactured in environments free from contaminants. The ability to detect contaminants in such environments can improve product quality, reduce losses attributable to fouled products, etc.

Moreover, the detection of certain chemicals and molecules in biological fluids is important for both diagnostic and therapeutic reasons.

Conventional sensors typically have employed sensor arrays that use heated metal oxide thin film resistors, polymer sorption layers on the surfaces of acoustic wave resonators, arrays of electrochemical detectors, and conductive polymers to detect specific target analytes in various fluids, including those in vapors, gases, and liquids. Clearly, however, a need still exists for alternative sensing technologies, particularly those that enable fast, inexpensive, efficient, and sensitive detection of one, several, or many different chemical and/or biological entities.

3. Definitions

When used in this specification, the following terms will be defined as provided below unless otherwise stated. All other terminology used herein will be defined with respect to its usage in the particular art to which it pertains unless otherwise noted.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

A “sensible” property is a property that can be detected.

In the context of chemicals (e.g., carbon dioxide, various hydrocarbons, oxides of nitrogen, etc.), the term “species” refers to a population of chemically indistinct molecules of the sort referred to, i.e., is a population of small molecules identified by the same chemical formula.

A “target analyte” refers to a chemical species to be detected or sensed.

### SUMMARY OF THE INVENTION

The object of this invention is to provide a new, patentable class of sensors that can be used to detect various chemicals and biological materials. At its core, the invention employs one or more chemically sorbent, substrate-free chemical sense elements capable of detecting or sensing the presence of one or more target analyte species in a gaseous or liquid environment. In some embodiments, the chemical sense elements are chemically sorbent, substrate-free polymeric solid composites comprised of (i) electrically conductive particles dispersed in electrically conductive relation in (ii) at least one structural polymer species. In other embodiments, the chemical sense elements are chemically sorbent, substrate-free conductive polymers. In other embodiments, the chemically sorbent, substrate-free chemical sense elements comprise electrically conductive polymers, which do not require (buy may nonetheless include) the inclusion of electrically conductive particles in order to conduct electricity. Representative examples of electrically conductive polymers include polyaniline, polythiophene, polyacetylene, poly(p-phenylene vinylene), and polypyrrole.

Regardless of whether the chemical sense elements are composites of structural polymers and electrically conductive particles or polymers that are themselves electrically conductive, the chemical sense elements of the invention have at least one sensible electrical property that can vary in the presence of a target analyte species. The chemical sense elements are formed to have at least two electrical leads in order to facilitate their integration with circuitry and associated hardware in functioning chemical sensor devices.

In some particularly preferred embodiments, chemical sense elements formed from chemically sorbent, substrate-free polymeric solid composites are those wherein the electrically conductive particles comprise an inorganic or organic electrical conductor, for example, carbon, copper, silver, or gold, representative examples of which include graphitized carbon, single- or multi-walled carbon nanotubes, carbon nanofibers, graphene, silver nanoparticles, and gold nanoparticles.

In composite-based chemical sense elements, preferred structural polymer species are those that are nonpolar, slightly polar, moderately polar, or highly polar under monitoring conditions. Illustrative examples of such structural polymer species include polydimethylsiloxane (PDMS), polyisobutylene (PIB), polyethylene (co-) vinylacetate (PEVA), polyepichlorohydrin (PECH), polycaprolactone (PCP), polyvinyl pyrrolidone (PVP), polyvinyl acetate (PVAC), polyvinyl alcohol (PVA), a polymer having intrinsic molecular porosity (PIM), hyperbranched poly{[bis(1,1,1-trifluoro-2-(trifluoromethyl)-pent-(Z/E)-4-ol)silylene]methylene} (HC), and hyperbranched poly{[bis(1,1,1-trifluoro-2-(trifluoromethyl)-pent-(Z/E)-4-ol)silylene]-[2-(1,1,1-trifluoro-2-(trifluoromethyl)-propan-2-ol)]propyne}.

Chemical sense elements are preferably formed as ribbons or threads by any suitable process, including casting, extrusion, drawing, or spinning.

As already described, a chemical sense element of the invention has a sensible electrical property, preferably resistance or impedance, which varies in the presence of a target analyte. In some embodiments, the sensible electrical property can be used to identify a target analyte signature in the presence of the target analyte, particularly when two or more chemical sense elements are used, each of which responds differently (in terms of sensible electrical property response) to the particular target analyte. Representative examples of target analytes that can be detected using one or more chemical sense elements according to the invention, alone or in conjunction with other chemical sensors, include various small molecule species, for example, chemical warfare agents, herbicides, pesticides, industrial chemicals, and explosives, and biomolecules, for example, those that are indicative of the presence of a pathogen, such as a biological warfare agent.

The invention also concerns integrating two or more chemical sense elements as an array in or on a flexible or solid support, such as fabric, paper, or plastic.

Another aspect of the invention relates to chemical sensors that include one or more chemical sense elements of the invention and circuitry in electrical communication with the electrical leads of the chemical sense element(s). The circuitry, including hardware and software, is configured to monitor for a change in sensible electrical property of the chemical sense element(s) and output and/or store signals reflective of the state of the sensible electrical property of the chemical sense element(s) over time.

In preferred embodiments, chemical sensors according to the invention also include not only power supplies (typically provided by one or more batteries), but also a microprocessor configured to control the energizing of the chemical sense elements and to analyze data from circuitry configured to detect changes in one or more sensible electrical properties of the chemical sense element(s) deployed in the chemical sensor, analog-to-digital converters, memory devices for storing data derived from the sense electrode circuits, as well as data and/or software for operating the sensor and for comparing results from the sense electrode circuits with data patterns representative of particular chemicals or biological materials, components that provide data logging and/or one- or two-way telemetry capability, etc., including RFID or other low-power radio transmitters or transceivers.

A related aspect of the invention methods of monitoring for and/or sensing target analytes in the environment in which a chemical sensor of the invention is stationed. The environment may be gaseous or liquid.

These and other aspects and embodiments of the invention are discussed in greater detail in the sections that follow. The foregoing and other aspects of the invention will become more apparent from the following detailed description, accompanying drawings, and the claims. Although methods and materials similar or equivalent to those described herein can be used in the practice or testing of the present invention, suitable methods and materials are described below. In addition, the materials, methods, and examples below are illustrative only and not intended to be limiting.

### BRIEF DESCRIPTION OF THE DRAWINGS

A brief summary of each of the figures is provided below.

FIG. 1 shows a conventional polymer composite chemiresistor (top right panel) that contains conductive particles and a sorbent polymer materials. In clean air (lower left panel) electricity conducts by a percolation path between electrodes, and when a chemical absorbs into the polymer (lower right panel), the polymer swells, separating the particles, and disturbing the percolation pathways.

FIG. 2 diagrams an unsupported polymer ribbon or thread chemical sense element that has access to air and therefore chemical analytes carried in the air from all sides. The thread or ribbon can therefore swell in all directions, allowing for faster absorption and desorption (recovery). The diagrams show such a chemical sense element in clean air (upper diagram) and after swelling in all dimensions when adsorbing or absorbing a chemical (lower diagram).

FIG. 3 shows a chemical sense element formed by solution casting a polymer-carbon (i.e., a polycaprolactone/carbon composite) film on a silicon wafer. A small, freestanding chemical sense element ribbon cut from the film to mimic a polymer composite thread can be seen to the right of the solution cast film.

FIG. 4 shows a close-up view of a chemical sense element ribbon chemiresistor (formed from a polycaprolactone/carbon composite) threaded through the eye of a needle (photo on left), as well as a close-up view of such a chemical sense element ribbon woven into a fabric patch (photo on upper right). The photo on the lower left shows the fabric patch from which the enlarged view shown in the upper right photo was taken. A coin (U.S. nickel) is shown for purposes of scale. The terminals of the chemical sense element ribbon can be removably connected to the circuitry of a chemical sensor, allowing such patches to be disposable such that they can be replaced from time to time.

FIG. 5 shows a response from a polycaprolactone/carbon composite ribbon as shown in FIG. 4 upon exposure to various concentrations of water and four different chemical vapors.

FIG. 6 shows the response of a chemical sensor using a composite chemical sense element exposed to two concentrations of methyl salicylate.

FIG. 7 shows various chemical sense element responses (resistance change relative to baseline resistance vs. time) to humidity, isooctane, toluene, ethanol, and DMMP exposure.

FIG. 8 shows the results of a principal component analysis (PCA) performed on training data (sensor responses) from a four polymer chemiresistor ribbon array according to the invention (see Example 3, below).

FIG. 9 shows photographs of two chemiresistor chemical sense elements according to the invention threaded through conductive fabric (photo on left) with wires for electrical connection to readout and close-up view of the chemiresistors (photo on right). A U.S. penny coin is shown for scale.

FIG. 10 shows a badge-sized chemical sensor system according to the invention for pollutant monitoring. A sorbent filled preconcentrator is mounted on a circuit board (approximately 2×2×1 inches) together with a pump, rechargeable batteries and a sensor flow cell. The polymer composite threads are mounted in the flow cell perpendicular to the flow. Each sensing thread has a two-point resistance measurement output linked to a microprocessor on the board. The circuit board controls the timing, temperature, and flow profiles. The badge is equipped with an optional USB output or a wireless readout for real-time sensing data.

FIG. 11 illustrates products that incorporate chemical sense element chemiresistor threads on patches, which contain metallic threads (for use as electrodes) and readout electronics for display or transmission.

FIG. 12 shows a Gore-Tex membrane having several structural polymer/electrically conductive particle composite chemical sense elements secured via an adhesive ring.

FIG. 13 shows a side view of two circular membranes of different diameter between which are sandwiched three structural polymer/electrically conductive particle composite chemical sense elements according to the invention.

FIG. 14 shows various embodiments of products that contain arrays of polymer-chemiresistor threads according to the invention (see Example 8).

FIG. 15 illustrates the filter system described in Example 9.

### DETAILED DESCRIPTION

### Polymer-Based, Conductance-Based Sensors

Conducting polymersⁱ: There are two main types of electronically conducting polymers: (1) the “organic metals”, those organic materials that are inherently conductive due to their electronic structure, typified by polyaniline, polypyrrole, polythiophene, and polyacetylene; (2) composites made from conventional, insulating organic polymer matrixes, loaded with conductive particles such as carbon or silver at sufficiently high levels to form continuous conductive pathways through the matrix. Films prepared from both of these categories allow straightforward (dc) resistance measurements of film properties, without large power requirements or complex circuits. Another type of resistive film based on ionically conductive polymeric materials is made using a host matrix through which ions can move readily, such as poly(ethylene oxide) (PEO), and a salt for which one or both components are mobile in the host, such as LiClO₄ in the case of PEOⁱⁱ. These materials are usually much more resistive and require more complex ac measurement circuitry, which can probe the ionic conductivity via capacitive coupling, rather than direct (Faradaic) electron transfer, may be required to obtain the best results from these materials.

Polymer compositesⁱⁱ: Conductive carbon or metal-loaded polymer composite-based chemiresistors are an inexpensive, easily fabricated matrix for sensor arrays. The conductive particles form electron transfer networks through the polymer films. Films can be made of any polymer with varied conductive particle concentration. The composite film resistance depends strongly on the concentration of the conductive materials and temperature.^{iii,iv,v}

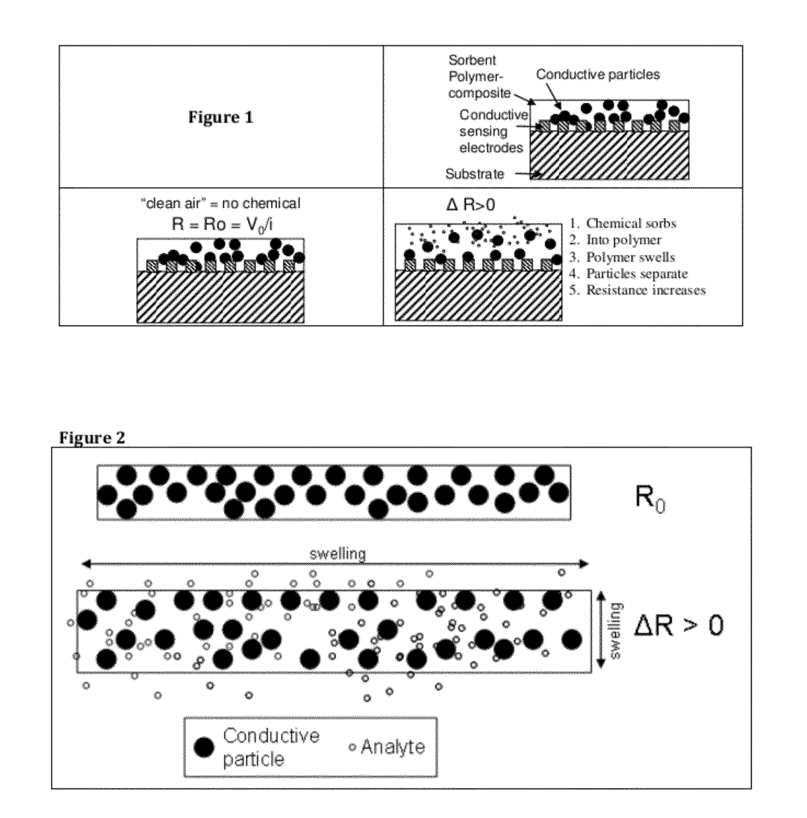

When a polymer/conductive particle composite expands its volume by thermal expansion or by swelling when absorbing a chemical, the electrical resistance increases due to a breaking of some of the conductive pathways through the film, and these changes can be very large if the polymer volume is changed close to the percolation threshold.^{iv,v} These composite films respond to different solvents depending on the particular solvent-polymer interaction, while the conductive particles only report the degree of swelling^{ii,iii} (e.g., see FIG. 1).

Among VOC-sensing techniques, polymer films are uniquely suited to small, low-power, low-cost sensors^{vi,vii}. All polymer/sorbent based detectors work with the same basic principle, only the transducer differs. Polymers are selected based on their ability to form stronger reversible chemical bonds (hydrogen bonds, van der Waals bonds, and dipole-dipole interactions) with the analyte rather than with interferents^{viii}. The amount of VOC that absorbs into the polymer depends on certain chemical properties of the polymer: for example, nonpolar polymers tend to absorb nonpolar analytes, while polar polymers tend to absorb polar analytes^{iii,ix,x,xi,xii}. It is possible to distinguish different VOCs from each other, by comparing the responses of several sensors^{xiii}; each constructed with a different polymer. Using pattern recognition algorithms in conjunction with multiple sensors in the array can mitigate remaining cross-sensitivities.

Hansen solubility parameters^{xi,xiv} (HSP) are one semi-empirical method of modeling and predicting the strength of the interactions between polymers and chemicals. When the solubility parameter of two liquids or a liquid and a polymer are close, they are highly miscible and likely absorb each other. The more chemical that is absorbed, the greater the measurable change in that material's chemical, physical or electrical properties, and in polymers, the more swelling that can occur. Hence, polymers that have similar solubility parameters to a chemical such as for example, methyl salicylate (MeS) (a chemical sometimes used to simulate CWAs) are likely to have a strong response to MeS. For example, MeS has a total HSP^{xi} value (δₜ) of ˜24.2, with the ability to form strong polar (δₚ=8) and hydrogen bonding (δₕ=13.9) interactions. In comparison, another aromatic compound, toluene, has weak polar interactions (δₚ=1.4) and weak hydrogen bonding capability (δₕ=2) with δₜ=18.2. One can predict that a polar polymer with a δₜ closer to 24 will sorb MeS better than toluene.

### Composite-Based Chemical Sense Elements

Chemical sense elements can be made from composites of structural polymers and electrically conductive particles. The conductive particles are suspended in the host polymer generally in the concentration range of 20-50% by weight conductive materials. The polymers are selected by their ability to provide high selectivity for adsorption or absorption of a particular analyte or class of analytes. Representative examples of suitable structural polymers and the rationale for their selection are provided in Table 1, below.

TABLE 1
Example polymers and selection rationale

| Polymer | Properties | Rationale/Peak sensitivity |
|---|---|---|
| Polydimethylsiloxane | Nonpolar siloxane polymer | Low polarity; commonly available |
| (PDMS) | | polymer; easy to cast and cross-link |
| Polyisobutylene (PIB) | Nonpolar polymer | Peakⁱ: Cyclohexane -Xylene; |
| | | Highest sensitivity to lowest polarity |
| | | chemicals |
| Polyethylene (co-) | Low Polarity polymer | Peakⁱ: Trichloroethylene; |
| vinylacetate (PEVA) | | Highest sensitivity to low-mid polarity |
| Polyepichlorohydrin | Mid-polarity, hydrogen-bond- | Demonstrated response to toxic |
| (PECH) | base | industrial chemicals^{xv} and chlorinated |
| | | solvents^{xvi} |
| Polycaprolactone (PCP) | Mid polarity, low hydrogen | Demonstrated high sensitivity^{xvii} to |
| | bonding. | Dimethyl methylphosphonate; δ^{xviii} |
| | | ~21-21.85 |
| Polyvinyl pyrrolidone | High polarity, strong hydrogen | Peakⁱ: Ethanol, Methanol - Water; |
| (PVP) | bonding | High sensitivity to polar hydrogen |
| | | bonding chemicals, e.g. alcohols and |
| | | water |
| Polyvinyl acetate (PVAC) | High polarity, strong hydrogen | Peak^{xix}: Alcohols- Water |
| | bonding | |
| Polyvinyl alcohol (PVA) | Strong hydrogen bonding | Peak^{i, xix}: Methanol - Water; e.g. PVA 88% |
| | | hydrolyzed is highly selective for water |
| Polymers with intrinsic | Mid polarity | Can be designed to have selective |
| molecular porosity (PIM)^{xx} | | response to target analytes |
| hyperbranched | Polar; hydrogen-bond acid | Demonstrated response to |
| poly{[bis(1,1,1-trifluoro-2- | | organophosphates, e.g., CWA's and |
| (trifluoromethyl)-pent- | | simulants |
| (Z/E)-4- | | |
| ol)silylene]methylene} | | |
| (HC)^{xxi} | | |
| hyperbranched | Polar; hydrogen-bond acid | Demonstrated response to |
| poly{[bis(1,1,1-trifluoro-2- | | organophosphates, e.g., CWA's and |
| (trifluoromethyl)-pent- | | simulants |
| (Z/E)-4-ol)silylene]-[2- | | |
| (1,1,1-trifluoro-2- | | |
| (trifluoromethyl)-propan- | | |
| 2-ol)]propyne} | | |
| (1STH157C)^{xxi} | | |

Suitable conductive materials for inclusion in composite-based chemical sense elements are those that can be readily mixed with the structural polymer species in order to form a substantially homogenous mixture of the composite material to ensure a substantially consistent distribution of the electrically conductive material within the structural polymer. Suitable conductive materials include graphitized carbon, single- and/or multi-walled carbon nanotubes, carbon nanofibers, graphene, silver nanoparticles, gold nanoparticles, and other inorganic conductive materials such as copper, aluminum, etc. Such materials can be purchased from commercial sources or manufactured using published techniques.

Chemical sense elements made from composites of structural polymers and electrically conductive particles can be prepared by any suitable method, including casting, deposition, extrusion, drawing, and spinning. Solutions of structural polymers are made in concentrations ranging from 0.5 to 5% by weight or volume with the added conductive particles preferably being homogenously dispersed by ultra sonication. Solution cast polymer-composite films can be dried at ambient or elevated temperature of up to 100° C. under atmospheric pressure. Subsequent annealing at elevated temperatures is optional. Ribbons are created by dissecting the polymer film into suitable lengths and widths. Good candidates for polymer composite ribbons show (1) good mechanical stability as an unsupported, freestanding film, and (2) good interaction (adsorption, absorption) with one or multiple of the targeted analytes for sensing.

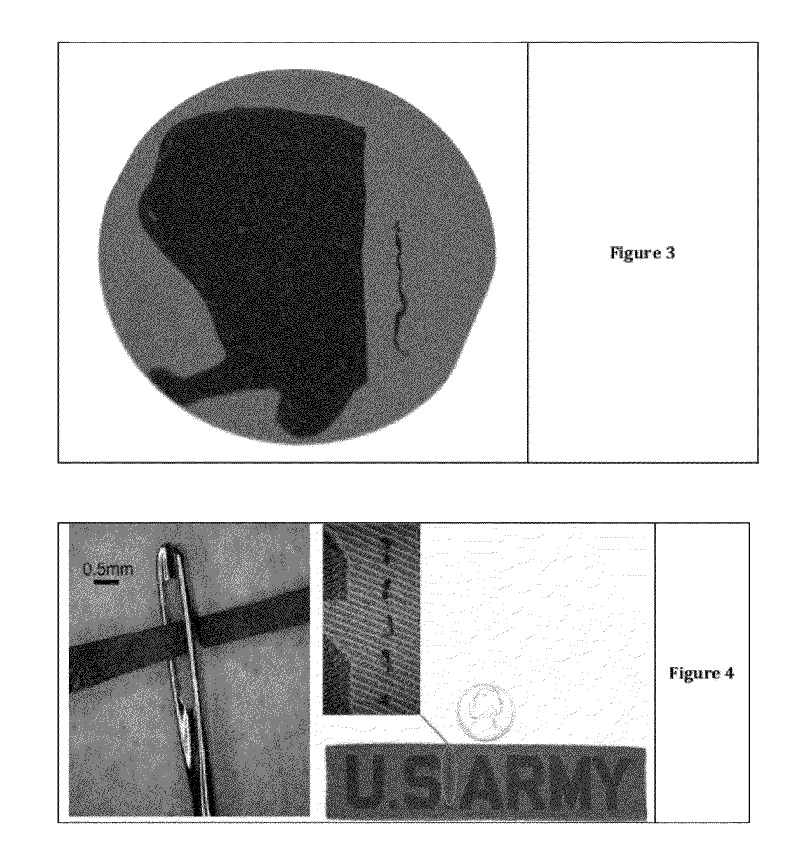

Chemical sense element ribbons can be fabricated, for example, by solution casting a structural polymer-carbon film onto a silicon wafer, followed by cutting small strips of the polymer-composite from the freestanding film. This representative process is shown in FIG. 3, which displays a cast polymer composite film and a narrow ribbon dissected from it. These ribbons show excellent mechanical stability compared to previously reported dip-coated supported polyester threads, and these ribbons best mimic an extruded polymer thread.

If a composite chemical sense element thread is to be manufactured by extrusion, the structural polymers may need to be melted, which requires a crystalline polymer or the plasticating of an amorphous polymer (transition from solid into a liquid without phase transformation). Polymers with a high glass transition temperature point (T_{g}) have generally good mechanical properties, but lower T_{g} may promote better interaction with the target analyte. Polymer composite ribbons produced from film deposition require the polymer to be soluble and the conductive additive to build a homogenous emulsion in the polymer solution.

Aqueous and non-aqueous solvents are suitable for the film deposition process. Liquid polymers are generally not suitable to form freestanding polymer composites with either manufacturing process (extrusion or film deposition). However, liquid polymers can be mixed with solid polymers to form mechanically stable, freestanding ribbons or threads. For instance, low molecular weight polyisobutylene (PIB), a viscous liquid at room temperature, may be mixed with a supporting polymer, such as PEVA, with a mass content of 10 to 70% PIB to form a freestanding film. The physical and chemical properties of the polymer blend are likely to be different from either constituent, therefore a valuable addition in a sensor array.

This invention requires that the material compositions used to manufacture a chemical sense element and associated circuitry in the chemical sensor maintain its physically integrity in the presence of anticipated environmental operating conditions and all anticipated species of target analytes in the environment of use for a duration equal to or greater than the desired life for the sensor. In some instances, it may be desirable to utilize at least two of the same chemical sense element in a chemical sensor to, for example, provide redundancy in the event that one such chemical sense element fails, in which event the redundant chemical sense element can provide the necessary signal for reliable operation of the sensor.

Chemical sense elements of the invention can be used in many ways. For example, they can be suspended as ribbons or threads, they can be placed adjacent to a single membrane or porous secondary support (which can protect one side or portion of the sense element adjacent to the physical support), and they can be sandwiched between two membranes or secondary supports (for better protection from liquids, for example), at least one of which is sufficiently porous to allow gas or vapor penetration. When suspended, a chemical sense element is ultimately connected to or suspended between two electrical leads or electrodes in the air or liquid environment where the target analyte(s) is to be detected. As will be appreciated, a suspended chemical sense element has the highest surface area available for chemical adsorption or absorption, resulting in faster possible response than for the same polymer on a substrate. A chemical sense element can also be threaded into a secondary substrate and connected electrically to appropriate circuitry for power and environmental measurement and/or monitoring.

In embodiments that employ one or more membranes or other secondary supports in conjunction with a chemical sense element, at least one of the membranes or secondary supports is a sufficiently porous support allowing air/chemical vapor/gas to pass through while blocking liquids. Chemical sense elements made of structural polymer/conductive particle composites, as well as electrically conductive polymers, are also porous, thus allowing fast (i.e., high flux) chemical transport (diffusion/convection) in and through the component. As will be appreciated, chemical sense elements swell in response to target analyte sorption, with the extent of swelling being related to the concentration and the strength of the target analyte-polymer interaction. A high flux and porous support allows for faster sensor response due to diffusion driving force from all sides, unlike silicon supported sensors. Chemical sense element ribbons and threads can be made from sheets, and precision cutting technologies such as lasers can be used to cut and trim the material to produce chemical sense elements with uniform, reproducible sensible electrical properties.

After a chemical sense element has been constructed, it may be integrated into a functional chemical sensor device, or into a component intended for use with such a device, for example, as part of a fabric patch, paper filter, or the like. In order to connect a chemical sense element to chemical sensor, the chemical sense element must be made part of an electrical circuit that will function when part of the chemical sensor. This is accomplished by providing for operable electrical connection of the chemical sense element (or array of a plurality of chemical sense elements) to other circuitry in the chemical sensor. Such connections can be permanent or detachable, depending on the particular application. Connection can, for example, be by way of attaching electrically conductive leads to suitable locations of a chemical sense element, including in at one or both ends of the particular chemical sense element and/or at locations along the length of the chemical sense element. Suitable types of electrical connection include conductive silver paste or silver epoxy connection to electrical leads, threaded or other secure yet detachable mechanical, electrical or magnetic connectors, and permanent connection (e.g., such as by soldering) of parts. Thus, monitoring of a chemical sense element can be made in multiple ways, including using at least two contact points on the chemical sense element for electrical connection to other circuitry, measuring each chemical sense element along various points along its length (which allows for example, for averaging of a response of the same sensor to reduce error or noise), and monitoring multiple independent chemical sense elements using, for example, pairs of contact points for each chemical sense element.

According to another embodiment, the invention provides a chemical sensor comprising at least one chemical sense element, as described above, and circuitry electrically connected to the chemical sense element(s), wherein the circuitry can detect a change in a sensible electrical property of the chemical sense element. The term “sensible electrical property,” as used herein, refers to any or a combination of detectable electrical parameters, including resistance, capacitance, inductance, impedance, phase angle, loss factor, dissipation, breakdown voltage, electrical temperature coefficient of an electrical property, Nernst current, impedance associated with ion conducting, open circuit potential, as well as an electrochemical property, an electronic property, a magnetic property, a thermal property, a mechanical property, or an optical property that can be detected or measured. Preferably, a sensible electrical property is selected from resistance or impedance.

Particularly preferred embodiments relate to chemical sense elements that are chemiresistors. The resistance measurement circuit can be any electronic circuit capable of measuring electrical resistance, such as an ohmmeter, multimeter, data logger, or custom current/voltage measuring device.^{xxii} As those will appreciate, resistance measurements can include a custom circuit on a printed circuit board (PCB) or application specific integrated circuit (ASIC). In some embodiments, the resistance measurement circuit can be formed on flexible printed circuit boards or fabric/textile circuit boards.

A chemical sensor according to the invention further includes a power source operatively connected to the chemical sense element(s). That energy source may also provide power to the shield layer, if present in the sensing electrode pair. Any suitable power source can be used. Depending on application, different power sources may be used. Suitable energy sources include one or more batteries, as well as electrical energy provided from a hardwired source (e.g., a generator, an electrical power grid, etc.). Autonomously powered supplies, for example, solar cells, piezoelectric or other movement-based power scavenging systems, etc. may also be used.

In preferred embodiments, chemical sensors according to the invention also include not only power supplies, but also a microprocessor configured to control the energizing of the chemical sense elements and to analyze data from circuitry configured to detect changes in one or more sensible electrical properties of the chemical sense element(s) deployed in the chemical sensor, analog-to-digital converters, memory devices for storing data derived from the sense electrode circuits, as well as data and/or software for operating the sensor and for comparing results from the sense electrode circuits with data patterns representative of particular chemicals or biological materials, components that provide data logging and/or one- or two-way telemetry capability, etc.

According to one embodiment, a chemical sensing element according to the invention is calibrated before use in a test environment. The calibration is preferably performed with a gas, vapor, or liquid mixture wherein the concentration of one of the target analytes is varied. During the calibration, one or more chosen sensible electrical properties versus the varying concentration of the target analyte is obtained. Such calibration data is preferably obtained for all target analytes to be detected by the chemical sensing element or chemical sensing element array. In the event that complex data is available, pattern-matching software (e.g., neural networks) can be utilized to correlate the response of a chemical sensing element array to each specific target analyte.

### Target Analytes

The target analytes to which a chemical sensing element according to the invention is responsive is almost limitless. As long as one can identify a chemical sensing element that interacts with a target analyte by adsorption, absorption, or another process that results in a change in a sensible electrical property of the chemical sense element, that target analyte can be detected using a sensor according to the invention. Indeed, the ability to change the polarity, hydrogen-bonding capabilities, or other chemical moieties used to manufacture chemical sense elements allows them to be used for the detection of various types of an incredible wide range of substances, including, for example, ketones, aldehydes, alcohols, amines, and organophosphorus and halogenated compounds.

Classes of target analytes include small molecules such as chemical warfare agents (e.g., nerve gases such as soman, sarin, mustard gas, etc.), herbicides, pesticides, industrial chemicals, explosives (e.g., TNT, nitro-compounds, etc.), and molecules that are considered simulants for such compounds; common solvents and volatile organic compounds (toluene, benzene, trichloroethylene, chloroform, acetone, ethanol, methanol, etc.); emission gases (CO₂, CO, NO₂, NO, SO, SO₂, etc.); and polycyclic hydrocarbons. Other target analytes that can be detected using chemical sense elements according to the invention include biological molecules such as peptides, lipids, sugars, nucleotides, polynucleotides, proteins, antibodies, whole cells, virus particles, bacterial cells, fungi, etc., and as such allow for the detection of pathogens, biological warfare agents, and the like.

As described, the invention is useful for detecting the presence of any number of different chemicals in a gas, vapor, or liquid phase, including toxic industrial chemicals, explosives and chemical warfare agents and simulants,^{i,ii,vii,ix,x}, and common pollutants and volatile organic compounds (VOC).^{i,iii} Example target analytes include but are not limited to the compounds listed in Table 2, below.

TABLE 2
Example target analytes

| | Solubility | |
|---|---|---|
| | parameter | |
| Chemical | (MPa^{1/2}) | Occurrence, reason for detection |
| Isooctane | 14.1 | VOC, Simulant for fuels |
| Toluene | 18.16 | VOC, Common solvent, Simulant for fuels |
| Trichloroethylene | 18.7 | Common solvent, Chlorinated chemical, |
| | | Pollutant from dry cleaners and industry |
| Acetone | 19.9 | VOC, Common solvent |
| DMMP | 22 | Nerve agent simulant |
| Nitrotoluene | 22.6 | Explosive simulant |
| Methyl salicylate | 24.2 | Mustard agent simulant used for chemical |
| | | suit and fabric breakthrough studies, e.g. |
| | | Man-in-Simulant Tests |
| Ethanol | 26.5 | Common solvent |
| Methanol | 29.6 | Common solvent, Used in deicing |
| | | operations |
| Water | 47.8 | Most common interferent |

As described, the chemical sense elements of the invention can be used for gaseous or fluid sensing applications. For instance, in waste water applications, the use of a hydrophobic chemical sense element array could be useful, such as crosslinked PDMS or polyurethanes. One benefit of such a chemical sense element array is that the chemical sense elements little resistance to the fluid flow when the array is placed into a moving liquid stream.

The invention will be better understood by reference to the following Examples, which are intended to merely illustrate the best mode now known for practicing the invention. The scope of the invention is not to be considered limited thereto.

### EXAMPLES

### Example 1

### High Sensitivity Sensor

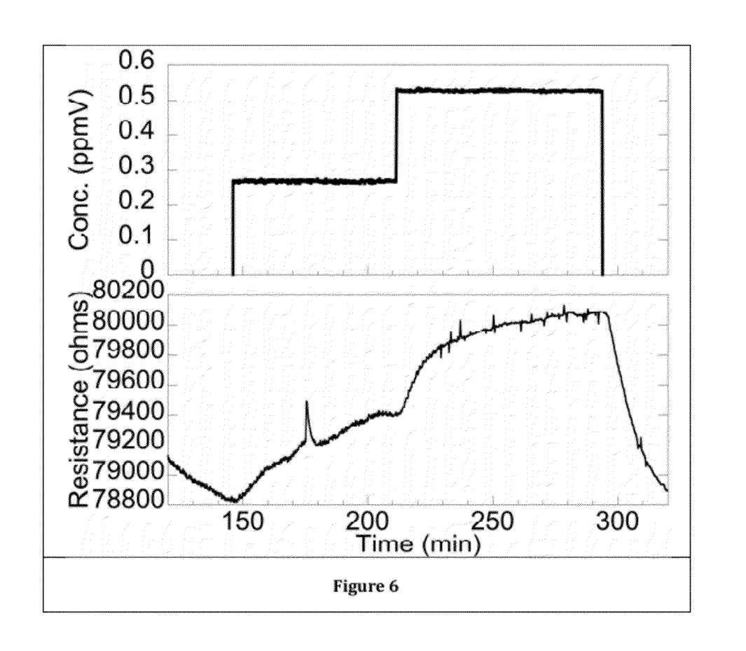

An example of high sensitivity of the chemical sense elements and sensors of the invention is shown in FIG. 6, where a chemical sense element ribbon was exposed to sub-ppmV (parts per million by volume) concentrations of methyl salicylate, a simulant used in the testing of chemical protective suits. The chemical sense element used was a PEVA-Carbon ribbon exposed to two concentrations of methyl salicylate (upper) at 20° C. The response of the smaller exposure can be extrapolated (using 3× peak-peak noise) to a limit of detection of 70 parts per billion by volume.

### Example 2

### Representative Chemical Sense Elements

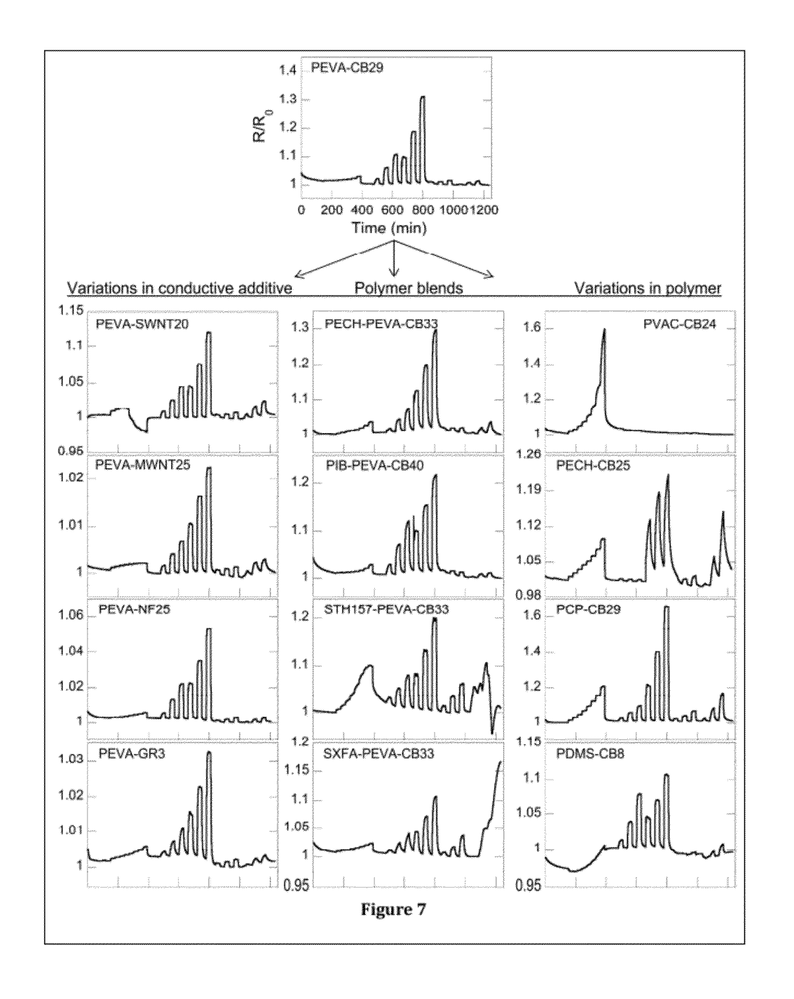

This example describes a number of different structural polymer/conductive particle composite chemiresistive chemical sense elements that can be produced by (1) changing the electrically conductive particle additive, (2) creating different polymer blends, and/or (3) varying the structural polymer. As an example, FIG. 7 shows a number of different compositions that can be used to build a sensor array. The test data shows a characteristic response for each individual polymer composite. Table 3 lists the composition of the polymer ribbons, the test results for which are shown in FIG. 7 as resistance versus time. An array of different ribbons can be built by varying the conductive additive (FIG. 7, column 1), produce polymer blends (FIG. 7, column 2), or use different polymers (FIG. 7, column 3). The response to each chemical is characteristic to each individual ribbon.

TABLE 3
Polymer/conductive composite ribbons

| Polymer/conductive composite | Abbreviation | Variable |
|---|---|---|
| Polyethylene (co-) vinylacetate/ | (PEVA-CB) | Conductive |
| carbon black | | additive |
| Polyethylene (co-) vinylacetate/ | (PEVA-SWNT) | |
| single wall carbon nanotubes | | |
| Polyethylene (co-) vinylacetate/ | (PEVA-MWNT) | |
| multi wall carbon nanotubes | | |
| Polyethylene (co-) vinylacetate/ | (PEVA-NF) | |
| nanofibers | | |
| Polyepichlorohydrin mixed with | (PECH-PEVA-CB) | Polymer |
| PEVA-CB | | blends |
| Polyisobutylene mixed with | (PIB-PEVA-CB) | |
| PEVA-CB | | |
| Polycarbosilane mixed with | (STH157C-PEVA-CB) | |
| PEVA-CB | | |
| Polyvinyl acetate/carbon black | (PVAC-CB) | Main |
| Polyepichlorohydrin/carbon black | (PECH-CB) | Polymer |
| Polydimethylsiloxane/carbon black | (PDMS-CB) | |

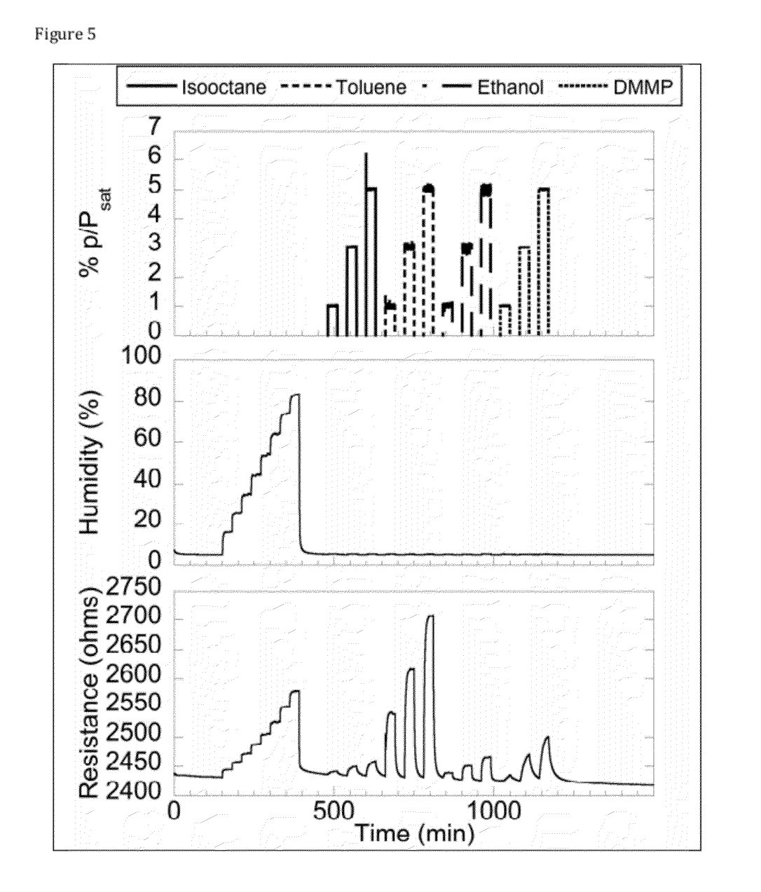

All ribbons listed in Table 3 were subject to the same test system sequence of chemical exposures as shown in FIG. 5, above (humidity, isooctane, toluene, ethanol, and DMMP). The matrix in FIG. 7 shows distinct response differences from the various ribbon polymer composites. A diversified detection response tremendously improves the results of any pattern recognition methods applied to the sensor array, for example, principal components analysis (PCA).

### Example 3

### Pattern Recognition (aka “Machine Learning”)

Sensor arrays, where the individual detector elements are sensitive to a number of chemicals, are sometimes called electronic noses. An electronic nose generally uses many different types of sensors^{xxiii} in order to mimic the olfaction (smelling) capabilities of mammals.^{xxiv} The receptors in the nasal cavities of mammals do not detect individual chemicals selectively, but use thousands of partially selective receptors that absorb inhaled chemicals in different ways. Each partially selective receptor may respond strongly, weakly, or not at all, to a specific chemical, resulting in a distinct pattern, which is sent to the brain for interpretation. The brain then determines if this “smell” pattern has been detected before and associates the chemical with a specific odor. The key is that different chemicals give the brain different patterns, and that these patterns determine what the brain thinks it smells. Electronic noses are designed to work similarly, with artificial intelligence, or sets of algorithms mimicking the decision making process of the brain.^{xxv}

These algorithms generally fall into two classes, quantification or classification. Quantifying algorithms such as linear or nonlinear regression techniques estimate the amount of an exposure (concentration or other level) based on comparison to training data or calculations derived from such data. Classification algorithms attempt to distinguish the class of an exposed chemical or mixture based again on comparison to training data or calculations derived from these data. Classification algorithms typically estimate the probability of detection and probability of correct identification of a detected sample.

A number of types of pattern recognition or machine learning techniques exist, a few of which are decision trees, neural networks, support vector machines, k-nearest neighbor, mean-clustering, principal component analysis, which are used for classification. For regression, approaches such as linear regressions, non-linear regression, neural networks, principal component analysis, and filters can be used.

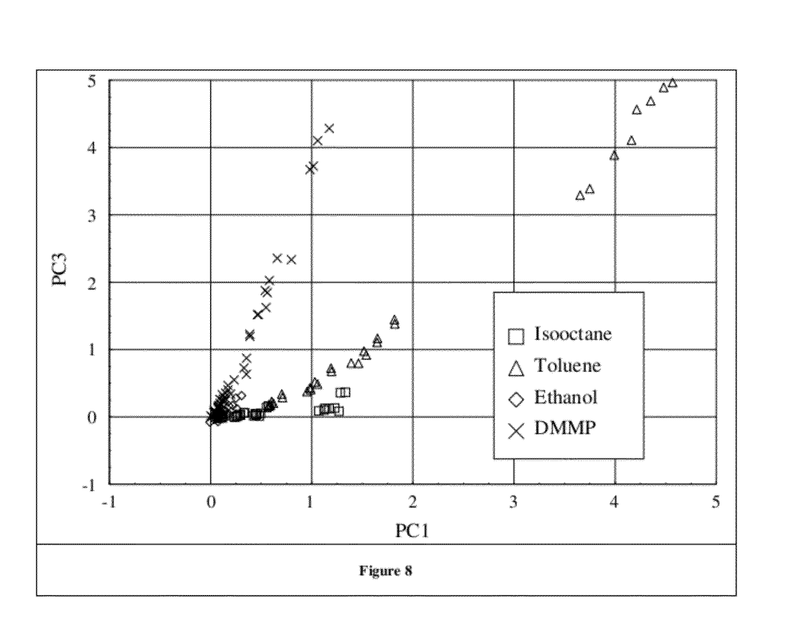

FIG. 8 shows the results of PCA performed on training data (sensor responses) from a four polymer chemiresistor ribbon array tested at 25° C. The four chemicals were varied in concentration (1-10% p/Pₛₐₜ) in background of various humidity levels (0-80%). PC1 and PC3 represent 43.1% and 30.3% of the total variance, respectively. The results show that DMMP, a chemical warfare agent simulant, can be easily classified from interfering chemicals representing common chemicals.

### Example 4

### Chemical Sensing Fabrics

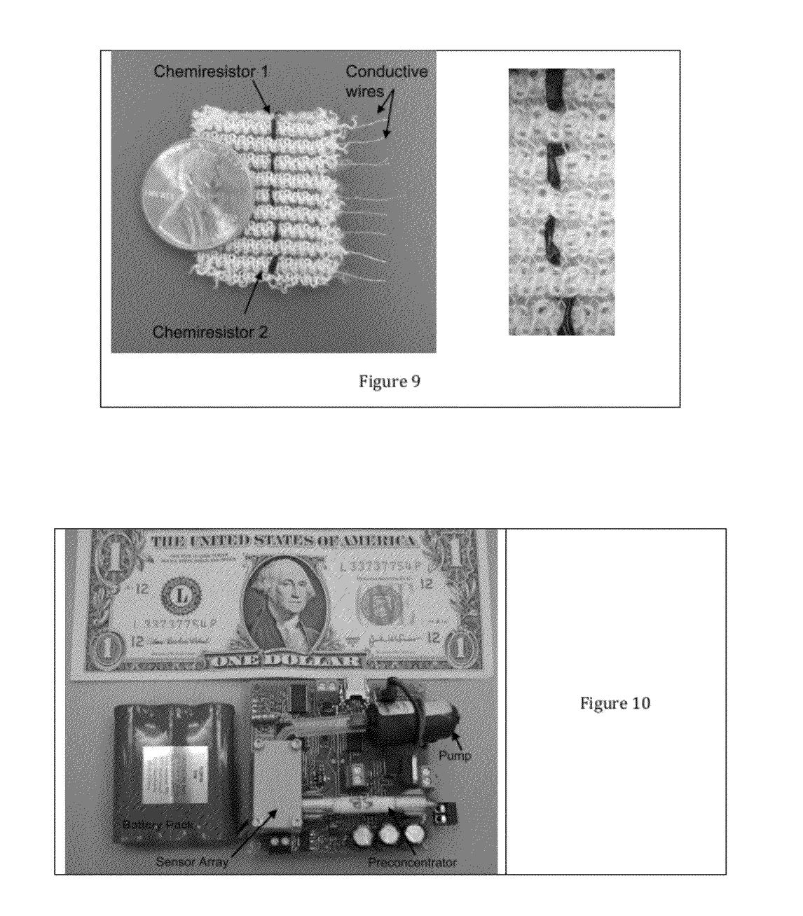

This example concerns freestanding chemical sense elements configured as ribbons or threads that can be woven into a fabric, e.g. patch, that can then be integrated with other circuitry to provide a functional chemical sensor. Fabrics containing metal or conducting fibers which can be used as electrodes or electrical contact points to other electronics can be used with the chemical sense elements (which here function as chemiresistors) by sewing the chemical sense elements ribbons or threads into the fabric, as shown, for example, in FIG. 9.

### Example 5

### Environmental Monitoring of Liquids

A representative application of chemical sense elements of the invention is in a badge-sized sensor for environmental monitoring. For improved sensitivities the sensor is equipped with a purge-and-trap preconcentrator. One such sensor is seen in FIG. 10, which shows the main sensor components on a printed circuit board, namely the sensor array, fluid pump, and preconcentrator. The sensor flow cell is equipped with a number of different chemical sense element chemiresistors configured as thread or ribbons to form a chemical sense element array that is aligned perpendicular to the fluid flow path through the flow cell. A representative array for environmental pollutant monitoring consists of the following combination of structural polymer/conductive particle composite chemical sense elements: PVAC-CB for direct humidity response; PEVA-NF for VOC detection (such as polycyclic hydrocarbons (PCHs) polyaromatic hydrocarbons (PAHs) and polychlorinated biphenyls (PCBs)); PDMS-CB for humidity discrimination and good response to VOC's; and STH157C-PCP-CB for more polar interferences discrimination (such as alcohols, ketones, and aldehydes).

### Example 6

### Lightweight, Low-Power, Chemical Sensing Detector Patch for Attachment to Outer Garments

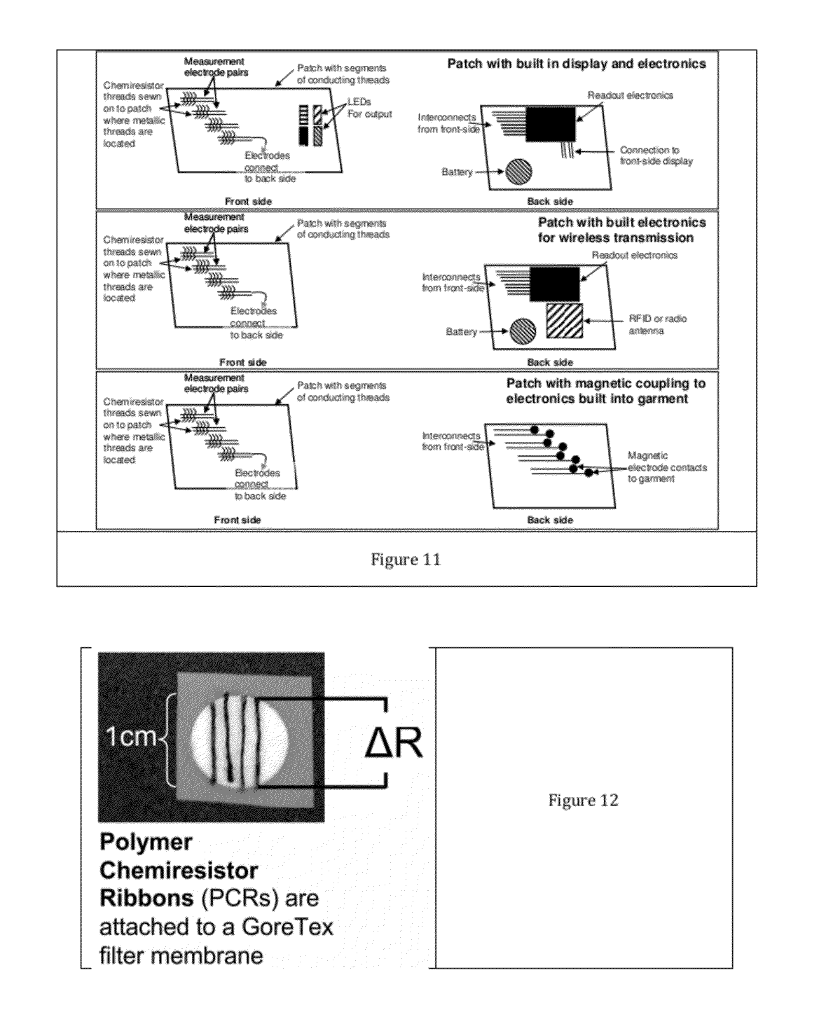

This example describes a chemical sensor that employs a plurality of different chemical sense elements configured as ribbons or threads that are woven as an array into a fabric patch, such as a patch that is worn on a uniform or other garment worn by a soldier, firefighter, policeman, healthcare worker, etc. Such patches can be made with wires or electrical conductors in appropriate locations, to act as measurement electrodes, contacts for circuitry, or antennae (see FIG. 11). The resulting patch is lightweight, requires only low-power, and has the capability to detect various chemicals, for instance chemical warfare agents, thereby providing a soldier with a hands-free chemical sensor. The patch can be sewn onto a uniform or, alternatively, it can be removably attached, for example, by Velcro or the like. If the electronics or situation requires, the system can be inexpensive enough to be disposable.

The circuitry can be made to be very thin, and be placed on the back side of the patch, as represented in FIG. 11. Preferably, the patch is attached to the wearer's outermost garment so as to maximize environmental exposure. In addition, the circuitry can be made to have a display on the front of the patch, in the form of a text, numeric, or other display, or warning lights, and/or can be made to transmit wirelessly to another device for readout and display, such as a blue-tooth enabled communication device. Data processing can be performed on the patch in a microprocessor or elsewhere by transmitting data gathered by the sensor array disposed on the patch.

An uncomplicated patch, as shown in the bottom panel of FIG. 11, could include only a plurality of chemiresistive chemical sense elements disposed as an array, where the chemical sense elements conduct electricity (for measurement) through magnetic contacts to corresponding contacts on the wearer's garment. The readout, display, or wireless system can then be integrated into the wearer's garments, resulting in a very low-cost, disposable sensing patch. Example products or users include:
Protective chemical sensor patches for warfighters—targets: chemical warfare agents, toxic chemicals
Protective sensor for domestic first responders (the US has nearly 2 million firefighters, EMS workers, and police officers)—targets: gases and volatiles from fires, illicit drug labs, industrial chemicals
Industrial workers, laboratory workers, chemists, scientists—targets: toxic industrial chemicals, common solvents, petrochemicals
Airport workers, gas station attendants, automotive repair workers who may be exposed to jet fuel fumes, during fueling operations

These sensors could then be used to warn the wearer of exposure to chemicals, providing time for them to don chemical protective suits. With circuitry to integrate exposure information, the device could also be used by industrial workers to measure total exposure or exposure dose, similar to radiation badges used by nuclear workers or radiologists.

### Example 7

### Membranes and Chemical Sense Elements

FIG. 12 shows a Gore-Tex membrane (other membranes can also be used) having several structural polymer/electrically conductive particle composite chemical sense elements secured via an adhesive ring. The adhesive ring provides an opening having a 1 centimeter (cm) diameter opening and allows the structural polymer/electrically conductive particle composite chemical sense elements to be applied directly to any desired surface, for example, the surface of a membrane or filter. In these chemiresistive sense elements, a resistance change, AR, correlates to chemical exposure concentration, and multiple different polymers together in the array provide information for pattern recognition.

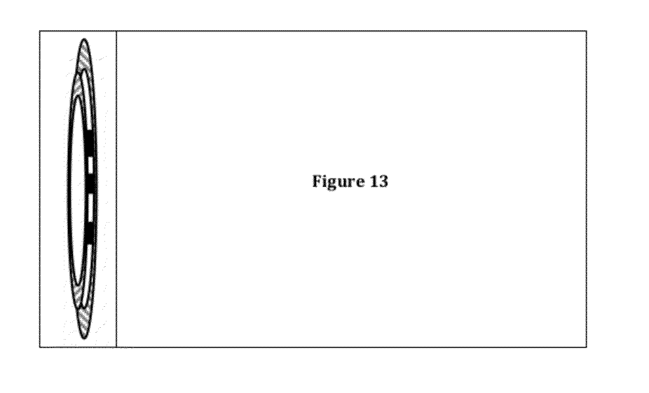

FIG. 13 shows two circular membranes, each of different diameter, sandwiching three chemiresistive sense elements according to the invention. The membranes enclose the sense elements and protect them from the liquid environment into which this sense element array is intended to be positioned for monitoring purposes. If desired, the sense element array can be further protected by using another membrane to cover the sensor elements, creating a sandwich of membranes encapsulating the sensor elements within.

### Example 8

### Sensing Patch for Chemical Suit

The United States (US) Departments of Homeland Security and Defense are testing next generation systems to protect U.S. military personnel from chemical threats. Some of these programs focus on Man-in-Simulant Testing (MIST), where methyl salicylate and live chemical warfare agents are used to test the effectiveness of chemical suits.^{xxvi,xxvii,xxviii} Methyl salicylate (MeS) is a typical test chemical used in chemical suit testing (ASTM method F2588-07^{xxix}).

Currently the suits are tested passively, that is, absorbent pads^{xxx} or tubes are used to collect samples while soldiers or mannequins (DOD's PETMAN program) wear the suits during MeS exposures. The protection offered by a protective ensemble is not determined solely by the material's properties, but also by the design of the suit. Zippers and openings at sleeves or the neck can contribute significantly to the reduction of the protective capability of a suit. To get an insight into the design-aspects of protective clothing, a “whole system” test is needed to reflect both the regional sensitivity of the body to chemical uptake and important garment design characteristics.

To better understand the suit lifetimes and weaknesses in the system, i.e., chemical break-through, small, unobtrusive, and light-weight sensors are needed to collect real-time data as the suits are used during tests. In a typical test, the volunteer wears 10 to 20 sorbent pads, placed at various locations on his/her body, and while wearing a protective suit, performs several tasks, such as walking, running, squatting, or twisting motions. The suit is exposed to chemicals and sorbent pads are later tested (chemical is thermally desorbed from each pad into a gas chromatograph with flame ionization detector). This procedure is the same for testing firefighter protective ensembles^{xxxi} and law enforcement and emergency medical services protective ensembles^{xxxii}. Real-time monitoring within these suits will provide material manufacturers, suit designers and researchers valuable data on where leaks or weak points occur in these ensembles.

A key requirement of this application is that the sensors do not interfere with air flow inside or through the suit or with the wearer's mobility. This significantly restricts the size and power of a viable sensor system. In addition the sensors must operate in the high relative humidity and elevated temperature environment of a person inside the suit. Microfabricated sensor arrays according to the invention can address this need.

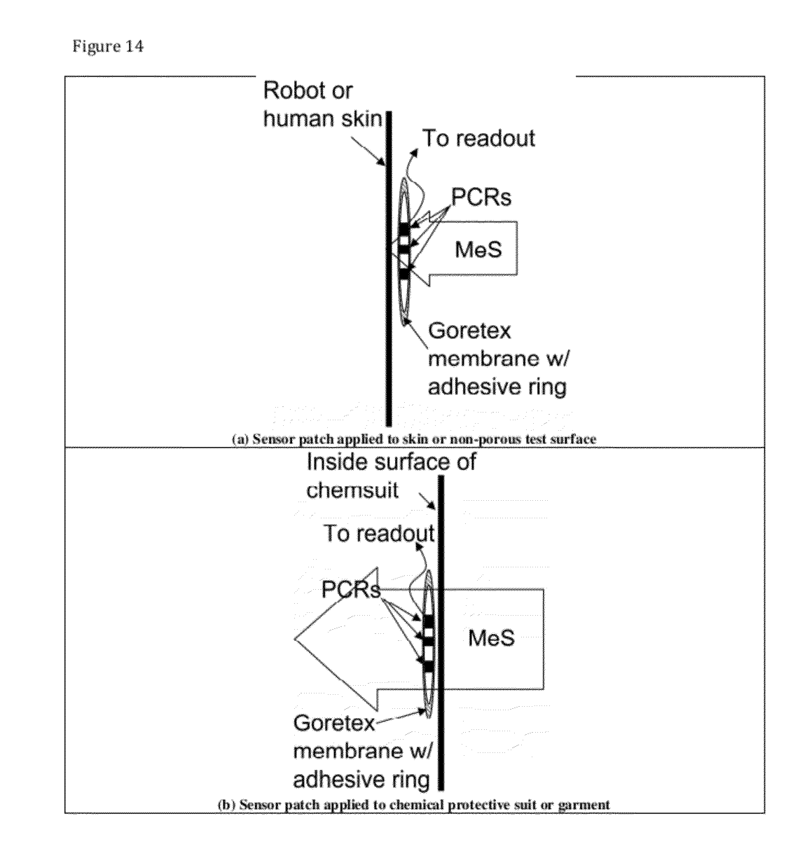

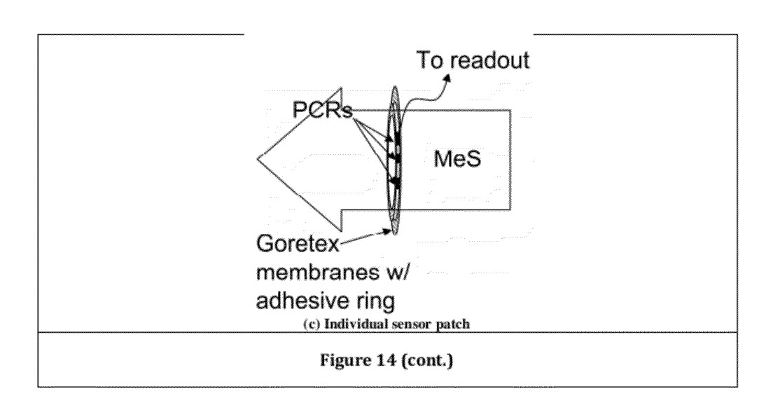

FIG. 14 shows various embodiments of products that contain arrays of polymer-chemiresistor threads, for example, as described in Example 7, above, and as shown in FIG. 12, which are thin enough to be used for MIST, PETMAN, and other similar test programs.

### Example 9

### Filters

Chemiresistive sense elements as described in Example 7, above, and shown in FIG. 12 can also be used in filter cartridges, for example, in a breathing apparatus or filter mask and filter materials. End of service life is a typical concern for manufacturers of filter cartridges. Such filters are used by military personnel, industrial workers, painters, etc. The problem with current technologies is that sensors emplaced within the filters may be too large or flow-restrictive and therefore interfere with filtering efficiency by producing channels for chemicals to by-pass the filter materials (channeling). The thin, flow-though design of some embodiments of the chemical sense elements of the invention allows for a sensor that does not create channels.

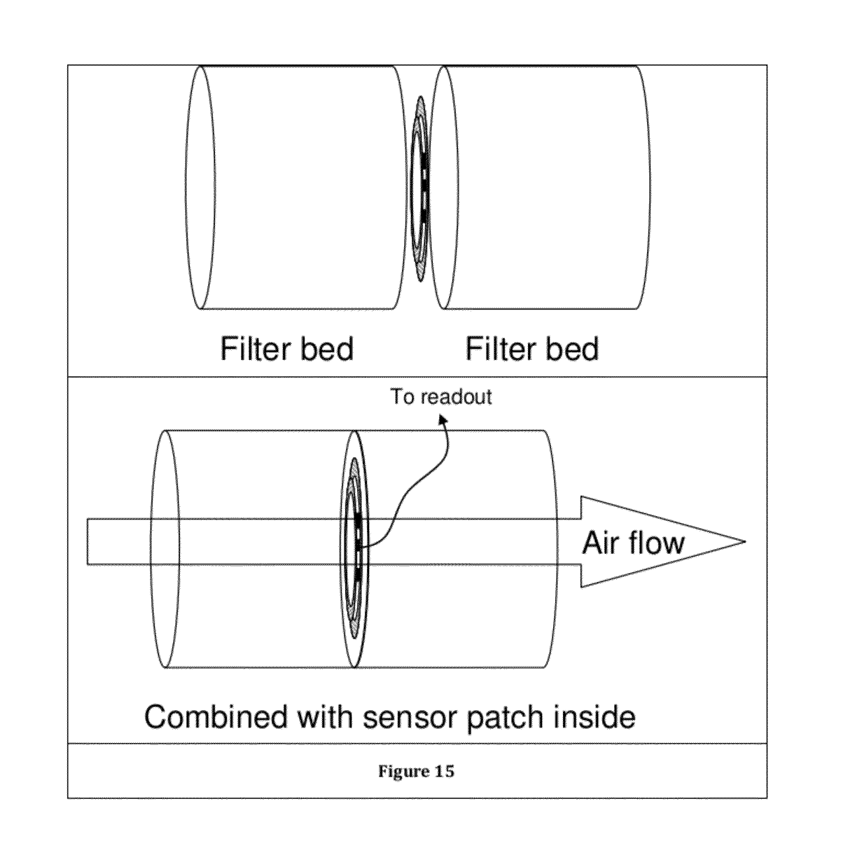

Thus, another application of the low-cost chemical sense elements and arrays of the invention is their use in air filters. Since the chemical sense element arrays are small and do not markedly interfere with air flow, they can easily be built into various filters, including HEPA filters or clean-room filters, where they can be used to monitor if potentially contaminating chemicals, biological agents, etc. are breaking through. Similarly, they can be integrated into a building's (or other enclosed structure's) HVAC system, where they can be used to protect, for example, from external chemical attacks by monitoring for chemical intrusion at air intakes. FIG. 15 illustrates a representative example of such a filter system. In this embodiment, a chemiresistive sense element as described in Example 7, above, and shown in FIG. 12 is positioned between two parts of a filter so that they are positioned directly in the airflow path.

Although the invention has been described with reference to the above Detailed Description and Examples, it will be understood that modifications and variations are encompassed within the spirit and scope of the invention. Accordingly, the invention is limited only by the appended claims.

All of the article, devices, and methods described and claimed herein can be made and executed without undue experimentation in light of the present disclosure. While the compositions and methods of this invention have been described in terms of preferred embodiments, it will be apparent to those skilled in the art that variations may be applied to the compositions and methods and in the steps or in the sequence of steps of the method described herein without departing from the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference in their entirety to the same extent as if each individual publication was specifically and individually indicated as being incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES CITED

ⁱS. V. Patel, M. W. Jenkins, R. C. Hughes, W. G. Yelton, A. J. Ricco, “Differentiation of Chemical Components in a Binary Solvent Vapor Mixture Using Carbon/Polymer Composite-Based Chemiresistors,” Analytical Chemistry, 72 (2000) 1532-1542.
ⁱⁱR. C. Hughes, W. G. Yelton, K. B. Pfeifer, and S. V. Patel, “Characteristics and Mechanism of Ion-Conducting Polymer Films as Chemical Sensors,” Journal of the Electrochemical Society, 148 (2001) H37-H44.
ⁱⁱⁱM. P. Eastman, R. C. Hughes, G. Yelton, A. J. Ricco, S. V. Patel, M. W. Jenkins, “Application of the Solubility Parameter Concept to the Design of Chemiresistor Arrays,” Journal of the Electrochemical Society, 146 (1999) 3907-3913.
^{iv}M. B. Heaney, “Resistance expansion temperature behavior of a disordered conductor-insulator composite,” Appl. Phys. Lett. 1996, 69, 2602-2604.
^{v}R. Viswanathan, M. B. Heaney, “Direct Imaging of the Percolation Network in a Three-Dimensional Disordered Conductor-Insulator Composite,” Phys. Rev. Lett. 1995, 75, 4433-4436.
^{vi}T. E. Mlsna, R. Mowery and McGill, R. A., “The Design of Aromatic Silicone Polymers and their Evaluation as Sorbent Coatings for Chemical Sensors,” Silicones in Coatings II, ISBN 09505319 44, 1998.
^{vii}Grate J W. Acoustic Wave Microsensor Arrays for Vapor Sensing. Chem. Rev. Mar. 28, 2000; 100(7):2627-48
^{viii}Grate J W. Hydrogen-Bond Acidic Polymers for Chemical Vapor Sensing. Chem. Rev. Jan. 26, 2008; 108(2):726-45.
^{ix}R. A. McGill, M. H. Abraham, J. W. Grate, “Choosing polymer coatings for chemical sensors,” CHEMTECH 24 (1994) 27-37.
^{x}S. V. Patel, T. E. Mlsna, B. Fruhberger, E. Klaassen, S. Cemalovic, D. R. Baselt, “Chemicapacitive microsensors for volatile organic compound detection,” Sensors and Actuators B, 96(3) (2003) 541-553.
^{xi}C. M. Hansen, Hansen Solubility Parameters, A User's Handbook, CRC Press, Boca Raton, Fla., 2000, Appx. A.1.
^{xii}A. F. M. Barton, CRC Handbook of Solubility Parameters and Other Cohesion Parameters, CRC Press, Boca Raton, Fla., 2nd edn., 1983.
^{xiii}Q. Cai, J. Park, D. Heldsinger, M.-D. Hsieh, E. T. Zellers, Vapor recognition with an integrated array of polymer-coated flexural-plate-wave sensors, Sens. & Act., B, Chem 62 (2000) 121-130.
^{xiv}Lange's Handbook of Chemistry, 14th ed., ed. John A. Dean, McGraw-Hill, New York, 1992, page 5-50 & 5-116.
^{xv}S. T. Hobson, S. Cemalovic, O. Katzenelson, S. Steele, A. L. Thibadeaux, “Synthesis and QSPR of Polymers in Sensor Applications,” Poster W113 presented at the Point Detection session, Chemical and Biological Defense Physical Science and Technology Conference, New Orleans, La., 17-21 Nov. 2008. http://www.seacoastscience.com/Downloads/Hobson(2008)%20Polymer%20QSPR%20poster.pdf ^{xvi}D. Rivera, M. K. Alam, C. E. Davis, C. K. Ho, “Characterization of the ability of polymeric chemiresistor arrays to quantitate trichloroethylene using partial least squares (PLS): effects of experimental design, humidity, and temperature,” Sensors and Actuators B 92 (2003) 110-120
^{xvii}A. R. Hopkins, N. S. Lewis, “Detection and classification characteristics of arrays of carbon black/organic polymer composite chemiresistive vapor detectors for the nerve agent simulants dimethylmethylphosphonate and diisopropylmethylphosphonate,” Anal. Chem. 2001, 73, 884-892.
^{xviii}B. S. Min, S. W. Ko, “Characterization of Segmented Block Copolyurethane Network Based on Glycidyl Azide Polymer and Polycaprolactone,” Macromolecular Research, Vol. 15, No. 3, pp 225-233 (2007)
^{xix} S. V. Patel, W. G. Yelton, and R. C. Hughes, “Effect of Hydroxyl Concentration on Chemical Sensitivity of Polyvinyl Alcohol/Carbon-Black Composite Chemiresistors,” Chemical Sensors IV, Proceedings Vol. 99-23, The Electrochemical Society, Inc., Pennington, N.J. (1999), pp. 163-167.
^{xx}N. B. McKeown and P. M. Budd, “Polymers of intrinsic microporosity (PIMs): organic materials for membrane separations, heterogeneous catalysis and hydrogen storage,” Chem. Soc. Rev., 2006, 35, 675-683.
^{xxi}HC and STH157 are materials fabricated from the following: R. A. McGill and E. J. Houser, U.S. Pat. No. 6,617,040, Sep. 9, 2003; R. A. McGill, E. J. Houser, and T. Mlsna. U.S. Pat. No. 6,630,560, Oct. 7, 2003; R. A. McGill and E. J. Houser.; U.S. Pat. No. 6,660,230, Dec. 9, 2003; E. J. Houser and R. A. McGill. United States Patent Application 2004/0058057 A1, Mar. 25, 2004
^{xxii}Hughes; R. C., Manginell; R. P., Jenkins; M. W., Kottenstette; R., Patel; S. V., “Apparatus for Sensing Volatile Organic Chemicals in Fluids,” U.S. Pat. No. 6,902,701, Jun. 7, 2005.
^{xxiii}H. T. Nagle, R. Gutierrez-Osuna, S. S. Schiffman, “The How and Why of Electronic Noses,” IEEE Spectrum, 35(1998) 22-34.
^{xxiv}J. E. Amoore, J. W. Johnston, M. Rubin, “The Stereochemical Theory of Odor,” Scientific American, 210 (2), 1964, pp. 42-49.
^{xxv}Gardner, J. “Review of conventional electronic noses and their possible application to the detection of explosives in Electronic Noses and Sensors for the Detection of Explosives,” (NATO Science Series II: Mathematics, Physics and Chemistry) J. Gardner and J Yinon eds. Kluwer Academic Publishers, Norwell, Mass. 2004.
^{xxvi}“Technical Assessment of the Man-In-Simulant Test (MIST) Program,” Standing Committee on Program and Technical Review of the U.S. Army Chemical and Biological Defense Command, National Academies Press, Washington, D.C., 1997. Available at: http://www.nap.edu/catalog.php?record_id=5936
^{xxvii}R. L. Barker, “A Review of Gaps and Limitations in Test Methods for First Responder Protective Clothing and Equipment,” final report presented to the National Personal Protection Technology Laboratory at the National Institute for Occupational Safety and Health, Jan. 31, 2005, p. 37.
^{xxviii}SBCCOM press release #02-31: “Vapor chamber tests chem./bio protective prototypes,” Jul. 5, 2002. Available at: http://www.natick.army.mil/about/pao/2002/02-31.htm
^{xxix}ASTM Method F2588-07 “Standard Test Method for Man-In-Simulant Test (MIST) for Protective Ensembles”
^{xxx}R. B. Belmonte, “Test Results of Level A Suits to Challenge By Chemical and Biological Warfare Agents and Simulants: Summary Report,” ERDC-TR, June 1998, SCBRD-EN, Aberdeen Proving Ground, MD, Appx. D, p. 44.
^{xxxi}“An Interim Summary Report for Firefighter Protective Ensemble (FFPE) Testing,” Prepared by: The Chemical Weapons Improved Response Program, Soldier and Biological Chemical Command Aberdeen Proving Ground, Maryland, April 1999. available at: http://www.chem-bio.com/resource/1999/cw_irp_ffpe_summary.pdf
^{xxxii}“An Interim Summary Report for Law Enforcement, and Emergency Medical Services Protective Ensemble Testing,” report prepared by The Chemical Weapons Improved Response Program, Soldier and Biological Chemical Command Aberdeen Proving Ground, Maryland, May 1999. available at: http://www.chem-bio.com/resource/1999/dp_le_ems_pe_summary_report.pdf

We claim:

1. A chemical sense element, optionally a chemiresistor, comprising:
a. a chemically sorbent, substrate-free polymeric solid composite comprised of (i) electrically conductive particles dispersed in electrically conductive relation in (ii) at least one structural polymer species; or
b. a chemically sorbent, substrate-free electrically conductive polymer,
wherein a sensible electrical property of the chemical sense element varies in the presence of a target analyte, and wherein the chemical sense element is formed to have at least two electrical leads.

2. A chemical sense element according to claim 1 wherein the chemical sense element is cast, extruded, drawn, or spun as a ribbon or thread.

3. A chemical sense element according to claim 1 that comprises a chemically sorbent, substrate-free polymeric solid composite, wherein the electrically conductive particles comprise an inorganic or organic electrical conductor, optionally carbon, copper, silver, or gold, and wherein the electrically conductive particles optionally are configured as graphitized carbon, single- or multi-walled carbon nanotubes, carbon nanofibers, graphene, silver nanoparticles, or gold nanoparticles.

4. A chemical sense element according to claim 1 that comprises a chemically sorbent, substrate-free polymeric solid composite, wherein the structural polymer species is nonpolar, slightly polar, moderately polar, or highly polar under monitoring conditions.

5. A chemical sense element according to claim 1 that comprises a chemically sorbent, substrate-free polymeric solid composite, wherein the structural polymer species is selected from the group consisting of polydimethylsiloxane (PDMS), polyisobutylene (PIB), polyethylene (co-) vinylacetate (PEVA), polyepichlorohydrin (PECH), polycaprolactone (PCP), polyvinyl pyrrolidone (PVP), polyvinyl acetate (PVAC), polyvinyl alcohol (PVA), a polymer having intrinsic molecular porosity (PIM), hyperbranched poly{[bis(1,1,1-trifluoro-2-(trifluoromethyl)-pent-(Z/E)-4-ol)silylene]methylene} (HC), and hyperbranched poly{[bis(1,1,1-trifluoro-2-(trifluoromethyl)-pent-(Z/E)-4-ol)silylene]-[2-(1,1,1-trifluoro-2-(trifluoromethyl)-propan-2-ol)]propyne}.

6. A chemical sense element according to claim 1 wherein the varying sensible electrical property is selected from the group consisting of resistance and impedance.

7. A chemical sense element according to claim 1 wherein the sensible electrical property can produce a target analyte signature in the presence of the target analyte.

8. A chemical sense element according to claim 1 wherein the target analyte is selected from the group consisting of a small molecule species, optionally a chemical warfare agent, an herbicide, a pesticide, an industrial chemical, or an explosive; and a biomolecule, optionally a biomolecule indicative of the presence of a pathogen, optionally a biological warfare agent.

9. A chemical sense element array, comprising a support and a plurality of chemical sense elements according to claim 1, wherein at least two of the chemical sense elements comprise different polymer species.

10. A chemical sense element array according to claim 9 wherein the support is selected from the group consisting of a flexible support, optionally fabric or paper, and a solid support, optionally a solid plastic support.

11. A chemical sensor, comprising:
a. at least one chemical sense element according to claim 1; and
b. circuitry in electrical communication with the electrical leads of the chemical sense element(s), wherein the circuitry can detect a change in sensible electrical property of the chemically sorbent, substrate-free polymeric solid composite.

12. A method of sensing a target analyte, comprising using a chemical sensor according to claim 11 to determine if the target analyte is present in an environment in which the chemical sensor is stationed.

13. A method according to claim 12 wherein the environment is a gaseous environment or a liquid environment.
